(19) United States
(12) Patent Application Publication
(10) Pub. No.: US 2003/0138358 A1
(43) Pub. Date: Jul. 24, 2003

(54) Method and device for microdosing the smallest amounts of liquid for biopolymer arrays

(76) Inventors: Heinz Eipel, Bensheim (DE); Markus Beier, Heidelberg (DE); Stefan Matysiak, Tettnang (DE)

Correspondence Address: Keil & Weinkauf, 1101 Conneticut Avenue NW, Washington, DC, 20036, US

(21) Appl. No.: 10/257,330

(22) PCT Filed: Apr. 6, 2001
(86) PCT No.: PCT/EP01/04000

(30) Foreign Application Priority Data
Apr. 10, 2000 (DE) 100 17 791.3

(51) Int. Cl. B01L 3/02

(52) U.S. Cl. 422/100; 436/180

(57) ABSTRACT

The invention relates to a process and an apparatus for the micrometering of extremely small quantities of liquid for the production of biopolymer arrays. The sample liquids to be analyzed are supplied by means of a supply device (1, 23), which can be connected to a stock of rinsing fluid (24). A reversible electric voltage (10) can be applied to the supply device (1, 23), enabling the electro-osmotic flow which arises to be used for the transport of the sample liquids onto a detection field (18).

The invention relates to a process and an apparatus for the micrometering of extremely small quantities of liquid for biopolymer arrays or biopolymer fields.

For the highly parallel analysis of biopolymers, such as, for example, nucleic acids, proteins or polysaccharides, use is made of micropolymer fields, also known as microarrays. For the production of such fields or arrays, very small biopolymer samples dissolved or suspended in liquids in the range from picoliters to nanoliters have to be applied in regular arrangements to substrate surfaces, for example to specimen slides. Conventional pipetting methods fail for such small quantities of liquid.

Since precise metering of the quantity to be transferred has hitherto been very difficult, precise metering is usually not carried out, and the quantities are transferred by means of an arrangement which involves mechanical contact, similar to a pen. However, such pens employed have only a limited liquid holding capacity, so that it is not possible to charge a multiplicity of substrate support surfaces with one pen filling. In order to increase the capacity of the pens employed, attempts have been made to provide these with notches or grooves in order for the pen to accommodate a larger quantity of sample substrate to be charged. Although this did enable the capacity of the pens to be increased, so that a larger number of biopolymer spots could be applied to a specimen slide with a single pen filling, cleaning of a pen of this design provided with grooves and slots was very difficult. Care must be taken that residues from prior sample charging runs are also removed from the grooves and slots expanding the capacity of the pens when a new biopolymer sample is to be applied by means of the pen to a specimen slide to be charged.

In order to keep measurement errors in analysis with the aid of such biopolymer arrays as small as possible, internal standards are usually used.

In view of the solutions known from the prior art and the disadvantages with which they are afflicted, it is an object of the present invention to charge biopolymer arrays with extremely small quantities of liquid in a simple and reliable manner.

We have found that this object is achieved in accordance with the invention by a process for the micrometering of extremely small quantities of liquid for the production of biopolymer arrays, in which the sample liquid to be analyzed can be supplied by means of a supply device, which can be connected to a rinsing fluid and to which a reversible electric voltage can be applied, enabling the electro-osmotic flow which arises to be used for the transport of sample liquid onto a detection surface.

The advantages which can be achieved with the process proposed in accordance with the invention are principally that a voltage which can be applied to the pipetting tip of the capillary tube accommodating the sample substrate enables extremely accurate metering of extremely small quantities of liquid at the point in time at which the pipetting tip has been positioned against the detection area of the respective specimen slide. If a plurality of capillary tube pipetting tips operated in parallel to one another are used through application of the voltage generating the transport of the sample liquid, individual biopolymer spots can be arranged on the detection surfaces of specimen slides inexpensively and quickly in a precise manner with achievement of highly accurate separations from one another.

In a further embodiment of the process proposed in accordance with the invention, application of an electric voltage to a drive capillary and the supply device causes a biopolymer to be drawn out of a sample stock and, after reversal of the voltage, the liquid to be metered to be dispensed. Accordingly, the dispensing of the sample liquid quantities and the production of the biopolymer spots on the surface of the detection field no longer requires components to be actuated mechanically within the cavity of the capillary tube.

According to a further advantageous embodiment of the process proposed in accordance with the invention, the electric voltage for the transport of the sample substrate is applied between the capillary head and the capillary space of the drive capillary. This allows the electric supply line to be fed into the upper part of the glass capillary, at the end of the glass capillary opposite to the pipetting tip.

According to a further advantageous aspect of the solution according to the invention, the pipetting tip of the capillary space can be moved in three directions. Besides movability of the pipetting tip in the X and Y directions above the detection field, the pipetting tip can be moved in the Z direction toward the surface of the detection field before a voltage which effects liquid ejection is applied to the contents of the capillary cavity.

In order to avoid losses of sample liquid and errors in applying the biopolymer pattern to the detection surface, reversal of the electric voltage, which effects ejection of the sample substrate from the capillary space of the capillary tubes, takes place with the pipetting tip positioned against the detection surface.

Finally, it is proposed in the process proposed in accordance with the invention for the metering of extremely small quantities of liquid that the drive capillary and its pipetting tip are connected by means of a valve to a buffer solution stored in a pressurized container, where the buffer solution used to generate an electro-osmotic pressure is a favorable buffer solution with a corresponding pH and ion concentration.

Finally, it is proposed to carry out electrophoretic deposition of charged biopolymer species on the specimen slide over a conductive layer to be applied on the surface of the specimen slide, i.e. to the detection surface. With this variant of the process proposed in accordance with the invention, analysis steps of subsequent analysis operations can be carried out even during the application and production of the biopolymer arrays.

According to the apparatus for the micrometering of extremely small quantities of liquid which is furthermore proposed in accordance with the invention, switching elements which reverse the voltage and are connected via an electric contact to the contents of a buffer container are integrated into the electric supply line for application of voltage to the drive capillary. By means of the apparatus proposed in accordance with the invention, extremely small quantities of liquid can be applied through the pipetting tip in the lowered state above a detection field of a specimen slide by simple reversal of the voltage due to the electro-osmotic flow in the sample substrate.

In order to guarantee continuous supply of the drive capillary with buffer solution, a flow resistance above the buffer solution container is incorporated in a branch of the drive capillary located behind a valve. Through suitable dimensioning of the flow resistance, bubble- and cavity-free supply of the drive capillary with buffer fluid can be achieved.

In an advantageous manner, the pipetting tip of a drive capillary or the pipetting tips of a plurality of drive capillaries can be moved above the detection field by means of an X/Y positioning unit of simple design, and the correct positions in which the biopolymer spots are to be applied to the detection surface can thus be set. Besides the movability of the pipetting tip in the X and Y directions, the positioning unit—for example a commercially available plotter—can also effect positioning of the pipetting tip in the Z direction toward the surface of the detection field.

The drive capillary and pipetting tip are advantageously made of glass or quartz.

The pipetting tip of the microcapillary is advantageously drawn out with a tip drawn out to a small diameter with a tip diameter in the range from 10 μm to 1000 μm. The diameter of the pipetting tip is particularly preferably in the range from 50 μm to 300 μm.

In order to ensure grounding, an electrical earth is provided between the pipetting tip and the drive capillary.

Finally, a connection for the generation of electro-osmotic flow is provided between the pipetting tip and the drive capillary, with a platinum wire electrode being accommodated in the capillary head of the drive capillary, and the further electrical connection of the end of the drive capillary being immersed into a buffer vessel provided with electric contacts. The voltage circuit at the drive capillary is thus interrupted merely by a pole reversal switch and can be interrupted or closed thereby in a frequency corresponding to the required charging frequency of the biopolymer spots onto the detection surface.

The invention is explained in greater detail below with reference to the drawing.

The single FIGURE shows a diagrammatic representation of the structure of an apparatus proposed in accordance with the invention for applying extremely small quantities of liquid in the picoliter to nanoliter range.

The pipetting tip 1 used to apply a sample liquid to the detection surface 18 of a specimen slide 9 is a glass capillary which is very inexpensive to produce, having a tip drawn out to a diameter of, for example, 200 μm. This capillary is connected at its end via a microhose to a drive capillary 2 of glass or quartz, as is usual in gas chromatography. A platinum wire electrode 3 for the production of an electric contact is inserted into the tube connection of the microhose. At the opposite end of the drive capillary 2 is a second electric contact 4, which projects into the contents of buffer solution accommodated in a buffer container 14. The fluid accommodated in the buffer container 14 is supplied continuously through a line branch 16, in which a flow resistance 13 is accommodated, so that the electric contact 4 is always in contact with the fluid in the drive capillary 2.

At the beginning of a pipetting operation, the pipetting tip 1 of a glass capillary is firstly moved over a waste container 7 using an X/Y positioning device, for example in the form of a commercially available graphic plotter or another X/Y positioning device. A valve 5 arranged upstream of the drive capillary 2 is subsequently opened briefly and so the drive capillary 2 together with the pipetting tip 1, positioned above the waste container 7, is filled continuously with fresh buffer solution, whose pH and ion concentration are set to a suitable value for the generation of electro-osmotic flow in the drive capillary 2, from a pressurized stock container 11 via a gas connection 6, and thus the pipetting tip 1 is simultaneously blown out over the waste container 7. The flow resistance 13 located in the said branch 16, for example in the form of a frit, causes a small quantity of buffer fluid to be forced into the buffer container 14, so that it is ensured that the drive capillary 2, which runs into the pipetting tip, is at all times charged with a continuously extending buffer stock.

A switching element 10, shown here in diagrammatic representation, is incorporated between the supply line 3 and the electric contact 4 to the buffer container 14. Two electric voltage sources, denoted by reference numerals 12a and 12b, are connected to the switching contacts of the switching element 10 and are grounded via an earth 17.

For drawing off of the biopolymer solution to be pipetted, which is provided in the biopolymer vessel 8, an electric voltage of suitable direction is applied to the two electric connections 3 and 4 via the switch 10 in order to generate an electro-osmotic flow in the backward direction in the drive capillary 2. At this point in time, the pipetting tip 1 is dipped, viewed in the Z direction, into the sample vessel 8, enabling sample substrate to be drawn up through the opening of the pipetting tip 1 in accordance with the applied voltage. When sufficient pipetting material has been drawn off from the biopolymer vessel 8, here, for example, a well of a microtiter plate, the automatic micropipetting system, i.e. the X/Y positioning device, positions the pipetting tip 1 over the substrate to be charged. The substrate can be, for example, a specimen slide 9, as frequently used in microscopy. A specimen slide surface 18, to which the individual biopolymer droplets emerging from the pipetting tip 1 are applied, is provided on the specimen slide 9. The detection surface 18 can also be a surface which chemically binds the biopolymer or interacts physico-chemically with the biopolymer. The application of the biopolymer spots to the specimen slide surface 18 takes place by means of the X/Y supply device, which in addition facilitates lowering of the pipetting tip 1 in the direction of the detection field 18. For this purpose, a reversed electric voltage is applied to the drive capillary 2 via the switch 10 for a selectable time, resulting in the liquid to be pipetted being forced out of the pipetting tip 1 through the electro-osmotic flow now running in the reversed direction and exiting onto the detection surface 18 of the specimen slide 9. The sample liquid can thereby be discharged either onto the detection surface 18 or into another vessel. As an alternative to the use of two voltage sources, a single voltage source with a corresponding switchover element can also be used, and other variants, for example of grounding, are entirely possible.

Through appropriate adjustment of the parameters affecting the electro-osmotic flow, such as principally the ion concentration and the pH of the buffer and the level of the applied electric voltage, the quantity of liquid dispensed during production of the individual biopolymer spots on the detection surface 18 of the specimen slide can be kept approximately constant. This enables a biopolymer pattern 19 which contains biopolymer spots arranged at regular separations 20 from one another both in the X and in the Y direction to be produced on the detection surface 18 of the specimen slide 9.

For acceleration and for electrochemical activation of the binding of the biopolymer spots to a suitable surface 18 of the specimen slide 9 which interacts chemically or physico-chemically with the biopolymer, an electric voltage of suitable polarity can, after contacting of the pipetting tip 1, additionally be applied between the connection 3 of the pipetting tip 1 and an electrically conductive surface on the specimen slide 9. This enables electrophoretic deposition of electrically charged biopolymer species even on the specimen slide shortly after their application, which is very beneficial for further analysis and sample evaluation.

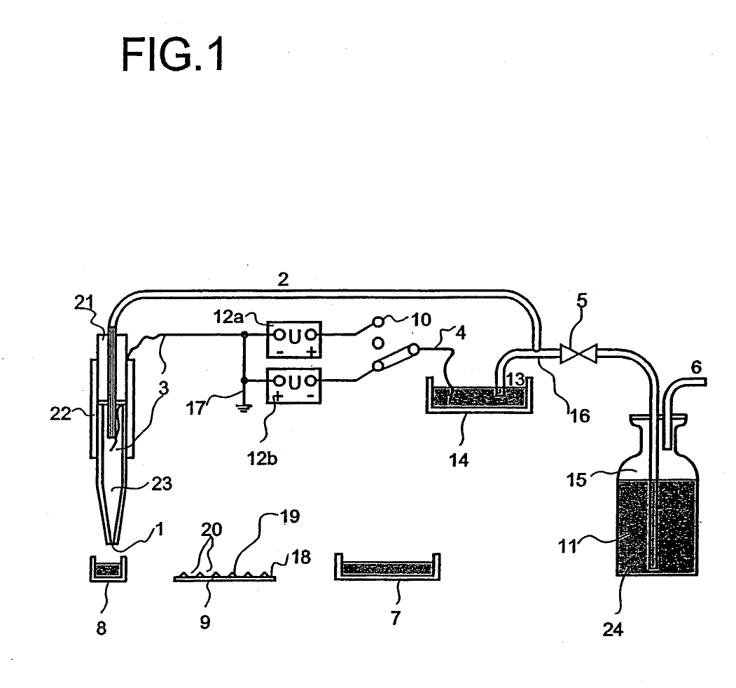

As can be seen in FIG. 1, the head of the drive capillary 2 is accommodated in a capillary head 21, which is itself surrounded by a mount 22, for example a short piece of hose. The glass or quartz pipetting tip 1, which has a cavity 23 into which the sample liquid to be pipetted is drawn up or, on reversal of the electro-osmotic flow, is ejected from the cavity 23, is admitted in a suitable manner into the mount 22. The pipetting tip 1, which is preferably made of glass, can have openings in the range from 10 μm to 1000 μm, with a diameter of from 50 μm to 300 μm preferably being formed at the pipetting tip opening 1.

### List of Reference Numerals

1 Pipetting tip

2 Drive capillary

3 Electric contact

4 Electric connection

5 Valve

6 Gas connection

7 Waste container

8 Biopolymer vessel

9 Specimen slide

10 Polarity reversal switch

11 Stock bottle

12a Electric voltage source+

12b Electric voltage source−

13 Flow resistance

14 Buffer container

15 Pressure line

16 T-piece

17 Grounding

18 Specimen slide surface

19 Biopolymer pattern

20 Separation of the biopolymer spots

21 Capillary head

22 Mount

23 Capillary cavity

24 Buffer solution

1. A process for the production of biopolymer arrays by micrometering of extremely small quantities of liquid, in which process samples to be analyzed can be supplied by means of a supply device, which can be connected to a stock container containing a rinsing fluid (24), wherein a reversible electric voltage can be applied between the supply device and a buffer vessel (14), enabling the electro-osmotic flow which arises to be used for the transport of the sample liquids onto a detection surface (18), wherein the drawing-off of a biopolymer from a vessel (8) and, after reversal of the voltage, the release of the biopolymer to be metered are effected by applying an electric voltage to a drive capillary (2) of the supply device.

2. A process as claimed in claim 1, wherein the supply device contains a capillary space (23) with a pipetting tip (1) and the pipetting tip (1) of the capillary space (23) can be moved in three directions.

3. A process as claimed in one of the claims 1 or 2, wherein reversal of the electric voltage, which causes sample liquid to exit from a capillary space (23) being part of the supply device is effected after a pipetting tip (1) being part of the supply device has reached the position against the detection surface (18).

4. A process as claimed in one of claims 1 to 3, wherein the supply device contains a pipetting tip (1) and the drive capillary (2) and the pipetting tip (1) can be supplied, by means of a valve (5), with a buffer solution, which is stored in a pressurized stock container and has a pH which is suitable for the generation of an electro-osmotic pressure and a suitable ion concentration.

5. A process as claimed in one of claims 1 to 4, wherein electrophoretic deposition of charged biopolymer species on a specimen slide surface (18) is effected by an electroconductive layer on the specimen slide surface (18).

6. An apparatus for the production of biopolymer arrays by micrometering of extremely small quantities of liquid with a supply device for the supply of sample substrates to be analyzed and with connecting lines for the connection of the supply device to a stock container, which contains a rinsing fluid (24), wherein the supply device contains a drive capillary (2), to which an electric supply line (3) is connected for application of a voltage between the supply device and a buffer solution container (14) via an electric contact (4), voltage-reversing switching elements (10) and electric voltage sources (12a, 12b) being connected to a electric supply line (3).

7. An apparatus as claimed in claim 6, wherein a flow resistance (13) is incorporated into a branch (16) of the drive capillary (2).

8. An apparatus as claimed in one of claims 6 or 7, wherein the supply device contains a pipetting tip (1) and wherein an X/Y positioning device which effects positioning of the pipetting tip (1) toward the surface of a specimen slide (9) is provided for positioning of the pipetting tip (1) in relation to a detection field (18).

9. An apparatus as claimed in one of claims 6 to 8, wherein the drive capillary (2) consists of glass or quartz.

10. An apparatus as claimed in one of claims 8 or 9, wherein the pipetting tip (1) is a tip drawn out from a glass capillary to a small diameter, the tip having a diameter in the range from 10 μm to 1000 μm.

11. An apparatus as claimed in claim 10, wherein the pipetting tip (1) is a tip drawn out from a glass capillary to a small diameter with a diameter in the range from 50 μm to 300 μm.

12. An apparatus as claimed in one of claims 8 to 11, wherein an electric earth is provided between the pipetting tip (1) and the drive capillary (2).

13. An apparatus as claimed in one of claims 8 to 12, wherein an electrical connection is provided between the pipetting tip (1) and the drive capillary (2) in order to generate an electro-osmotic flow, where a platinum wire electrode (3) in a capillary head (21) being part of the supply device and a further electric contact (4) at the end of the drive capillary (2) dip into a buffer vessel (14) provided with electric contacts.
